(19) United States
(12) Patent Application Publication
(10) Pub. No.: US 2002/0018994 A1
(43) Pub. Date: Feb. 14, 2002

(54) CONTINUOUS IN VITRO EVOLUTION OF OLIGONUCLEOTIDES

(76) Inventor: ASHER NATHAN, BEIT SHEMESH (IL)

Correspondence Address: FITCH EVEN TABIN & FLANNERY, 135 SOUTH LASALLE STREET, SUITE 900, CHICAGO, IL, 606034277

(21) Appl. No.: 09/403,433

(22) PCT Filed: Apr. 20, 1998
(86) PCT No.: PCT/IL98/00186

(30) Foreign Application Priority Data
Apr. 21, 1997 (IL) 120714

(51) Int. Cl. C12Q 1/68; C07H 21/02; C07H 21/04; C12P 19/34

(52) U.S. Cl. 435/6; 435/91.2; 536/23.1

(57) ABSTRACT

The present invention concerns a method and kit for carrying out in vitro evolution of a catalytic nucleic acid seuqences in a continuous manner, wherein the evolving nucleic acid sequences are DNA sequences.

### FIELD OF THE INVENTION

The present invention concerns in vitro evolution of catalytic oligonucleotides. More specifically, the present invention concerns a method and kit for continuous in vitro evolution wherein template-directed ligation of oligonucleotides is made to evolve in a continuous manner in a test tube.

### BACKGROUND OF THE INVENTION

Darwinian evolution may be made to operate in vitro by subjecting a population of informational macromolecules to repeated rounds of selective amplification and mutation. The first extracellular Darwinian evolution experiment employed variants of bacteriophage genomic RNA that were amplified based on their ability to serve as a substrate for the replicase protein. Evolution was carried out in a continuous manner by serial transfers of the RNAs to successive reaction vessels. In recent years, in vitro evolution techniques have been generalized to allow selective amplification of almost any nucleic acid molecule, including those that have a catalytic function, which are known in the art as “ribozymes” (Kumar, P. K. R. and Ellington, A. D., FASEB J, 2:1183 (1995)). However, unlike the evolution experiments, the evolution of molecules having catalytic function has been carried out in a stepwise rather than continuous fashion. Stepwise evolution requires frequent intervention by the worker at successive steps of catalysis for negative and positive selection, selective amplification, and mutation, and thus proceeds at a rate which is hundreds of times slower than would have been possible with continuous evolution.

Continuous evolution is disclosed by Wright, M. C. and Joyce, G. F., (in Press). This publication concerns a method for in vitro evolution of catalytic RNAs (i.e. ribozymes) or other oligonucleotides which are capable of condensation of 3′-hydroxyl of another oligonucleotide and the catalytic molecules' own 5′-triphosphate, generating a 3′,5′-phosphodiester linkage and releasing a pyrophosphate, which activity will be termed hereinafter “ligation”. The RNA sequence to which the evolving catalytic RNA condenses is a sequence complementary to a DNA promoter. This RNA sequence will be termed hereinafter as “RNA promoter”. Reverse transcription of the RNA molecules results in complementary DNA molecules. The RNA/DNA hybrid molecules are then treated with RNase H, capable of digesting the RNA strand in an RNA/DNA hybrid. DNA molecules which were reversed transcribed from catalytically active RNA contain one strand of the promoter element at their 3′ end, while DNA molecules reversed transcribed from non-catalytically active RNA do not contain such a promoter. In the presence of the complementary, non-template strand of the promoter a DNA construct having a double-stranded functional promoter is formed which is transcribed to an RNA transcript in the presence of T₇ RNA polymerase. Thus only DNA reverse transcribed from catalytic RNA gives rise to RNA transcripts while DNA reverse transcribed from non-catalytic RNA does not form new RNA transcripts as it lacks a promoter. The transcript produced from the former (which does not contain the promoter sequence) is capable of condensing again with the RNA promoter and thus being amplified again and again.

By such a scheme only catalytic RNA molecules capable of ligating to an RNA promoter are continuously multiplied while non-catalytic RNA molecules are digested by the RNase H. Since during the reverse transcription from RNA to DNA some mutations are inserted, the catalytic RNA molecule is capable of evolving in a continuous manner with no need for separate steps of synthesis, selection, amplification and mutation and the in vitro evolution process can continue indefinitely as long as RNA promoters, polymerases and reverse transcription proteins, non-template strands of DNA promoters and nucleic acid monomers are made available.

The drawback of the above continuous in vitro evolution scheme stems from the fact that two conflicting reactions take place simultaneously in the test tube: one reaction is the condensation (i.e. ligation) of the evolving catalytic RNA molecule to its RNA promoter and the second reaction is the reverse transcription of the RNA to an RNA/DNA hybrid which is not active. The rate of these two conflicting reactions may determine the fate of a catalytic RNA molecule. This is due to the fact that even if an RNA molecule is catalytic, it may be eliminated before it has a chance to ligate to the RNA promoter. Thus catalytically active RNA molecules may be undesirably eliminated from the evolving mixture simply because the reverse transcription proceeded at a higher rate than that of the ligation reaction, and subsequent digestion by RNase H. This elimination is especially problematic where initially the evolving catalytic RNA molecules ligate at a slow rate and, due to digestion of RNA strands by RNase H, may eventually bring about complete disappearance of all RNA molecules from the mixture notwithstanding the fact that some had catalytic potential.

It would have been highly desirable to provide a method for continuous in vitro evolution which would reduce or avoid the undesired elimination of catalytically active molecules in the reaction mixture.

### SUMMARY OF THE INVENTION

In accordance with the present invention a continuous in vitro evolution method is provided in which all evolving molecules, which are capable of catalytic activity, remain intact until the catalytic activity actually takes place, which method utilizes DNA sequences, as the evolving oligonucleotides.

It has been reported that it is possible to construct DNA oligonucleotides made essentially of DNA which feature catalytic activity. Such catalytic DNA molecules will be referred to herein as “dinozymes” (Breaker, R. R., and Joyce, Chemistry and Biolog;, 2(10):655-660 (1995)).

Thus the present invention provides a method for continuous in vitro evolution for obtaining oligonucleotides possessing a catalytic activity of ligation, splicing, or gap filling the method comprising:

(a) preparing an oligonucleotide mixture of essentially deoxy-oligo-nucleotides, comprising a plurality of different species of oligonucleotides being candidates for such having said catalytic activity, each species having a different sequence than the other oligonucleotides species;

(b) contacting said mixture with a reagent system comprising:

(b1) oligonucleotides which are substrates for the ligation, splicing, or gap filling catalytic activity, being essentially deoxy oligonucleotides, comprising a functional promoter,

(b2) RNA polymerase,

(b3) ribonucleotide triphosphates of U, A, C and G bases,

(b4) primers for reverse transcription,

(b5) reverse transcriptase,

(b6) deoxy-nucleotide triphosphates of A, T, C and G bases, and

(b7) RNase H, under conditions permitting:

(i) hybridization of complementary oligonucleotides,

(ii) cis ligation, splicing or gap filling, and

(iii) enzymatic activity of RNA polymerase, reverse transcriptase and RNase H;

whereby the reaction mixture becomes enriched with oligonucleotides having said catalytic activity.

The present invention further concerns a kit for carrying out the above method comprising:

(a) an oligonucleotide mixture of essentially deoxy-oligonucleotides, comprising a plurality of different species of oligonucleotides being candidates for such having said catalytic activity, each species having a different sequence than the other oligonucleotides species; and

(b) a reagent system comprising:

(b1) oligonucleotides which are substrates for the ligation, splicing, or gap filling catalytic activity, being essentially deoxy oligonucleotides, comprising a functional promoter,

(b2) RNA polymerase,

(b3) ribonucleotide triphosphates of U, A, C and G bases, and

(b4) primers for reverse transcription,

(b5) reverse transcriptase,

(b6) deoxy-nucleotide triphosphates of A, T, C and G bases, and

(b7) RNase H.

The term “ligation” refers to a reaction where the 3′-hydroxyl of the catalytic oligonucleotide is condensed to the 5′-triphosphate of the substrate oligonucleotide generating a 3′-5′-phosphodiester linkage and releasing pyrophosphate.

The term “gapfilling” refers to a reaction where one or more nucleotides are added to fill a gap between a catalytic oligonucleotide and a substrate oligonucleotide and thus the two oligonucleotides become physically connected.

The term “splicing” refers to a reaction typical of Group I ribozymes where catalytic oligonucleotides condense to the substrate similarly as defined in “ligation” above but instead of release of a pyrophosphate a short stretch of nucleic acid sequence is released.

The term “mixture of essentially deoxy-oligonucleotides” refers to a plurality of oligonucleotides species which are to be evolved by the method of the invention. All oligonucleotides having the same sequences are termed “species”. In the mixture each species has a different sequence than the sequence of the other species. The sequences can be prepared utilizing a DNA synthesizer and may be completely random or alternatively may be semi-random or “dopped”, i.e. each sequence features X% identity to a sequence of a known dinozyme while (100-X)% of each sequence is random. It is possible to construct sequences so that the semi-randomness or randomness is evenly distributed along the whole sequence of the oligonucleotide, or alternatively, that some regions of the oligonucleotide remain conserved (i.e. identical in all the oligonucleotides species present in the mixture) while the other regions are either random or semi-random. Preferably, the sequence of all the oligonucleotides in the mixture comprises a conserved 3′-end for hybridizing to the non-template strand of a promoter (as will be explained hereinbelow) and a conserved 5′-end which, when transcribed to RNA, is capable of hybridization to a DNA primer for reverse transcription purposes. The oligonucleotides are essentially made of deoxy-nucleotides and may comprise also non-naturally occurring deoxy-nucleotides such as ionosine and the like. In the initial reaction mixture it is also possible to use deoxy-oligonucleotides having several non-deoxy nucleotides inserted therein.

The mixture is then brought into contact with a reagent system capable of supporting continuous in vitro evolution, under conditions allowing ligation, splicing, gap filling, hybridization and enzymatic activity of the various enzymes which conditions are generally well known in the art.

The reagent system comprises the following:

(a) A substrate oligonucleotide, to which the evolving catalytic oligonucleotide should become attached by ligation, splicing, or gap filling, which substrate is composed essentially of deoxy-nucleotides, but may comprise also non-naturally occurring nucleotides as well as some ribo-nucleotides, for example the last nucleotide may be a ribo-nucleotide. The substrate oligonucleotide comprises a functional promoter. The substrate may, a priori, comprise a double-stranded promoter or alternatively the double-stranded promoter may be formed in the reaction mixture by providing both the template and the complementary non-template strands of the promoter which can then hybridize in the test tube. Alternatively, the oligonucleotide substrate may be a single-stranded oligonucleotide capable of folding to loop and thus forming a functional double-stranded promoter. The non-template strand of the double-stranded promoter should preferably also comprise an additional sequence complementary to the 3′-end of each evolving oligonucleotide species in the mixture, (as indicated above, said 3′-end should preferably be a conserved sequence for the purpose of said hybridization). The function of this additional sequence is to bring together the promoter and the evolving catalytic oligonucleotide by hybridization thus requiring only the catalytic activity (ligation, splicing, or gap filling) in order to form a functional construct capable of being transcribed. Such a ligated construct will be termed hereinafter “functional construct”.

(b) RNA polymerase, capable of producing (in the presence of ribonucleotides) an RNA transcript from the functional construct. An example of such a polymerase is the T₇ RNA polymerase. Even if catalytic reaction is slow, the catalytic oligonucleotides remain functional in the reaction mixture and are not eliminated, contrary to the method of Wright and Joyce.

(c) ribonucleotide triphosphate of A, U, C and G bases, which can include naturally or non-naturally occurring bases such as IsoG and IsoC.

(d) Primers for reverse transcription which are typically DNA sequences 10 to 50 nucleotides long capable of hybridizing to the RNA transcript and being elongated by reverse transcriptase to form a complementary DNA strand. The primers for reverse transcription hybridize with the 3′ of each RNA transcript, transcribed from the 5′ of the oligo-nucleotides in the original mixture. Thus, as explained above, all species of the original mixture should preferably have a conserved 5′-end for the purpose of hybridization to said primers.

(e) Reverse transcriptases, for example the AMV and the MMLV reverse transcriptases;

(f) Deoxy-nucleotide triphosphate of A, T, C and G bases (both naturally and non-naturally occurring) which in the presence of appropriate enzymes can give rise to an RNA/DNA hybrid based on the RNA transcript.

(g) RNase H which is capable of digesting the RNA strand in the RNA/DNA hybrid to give rise to a catalytic DNA molecule which can again become attached to a promoter substrate and re-enter the continuous in vitro evolution so that it can be amplified again and again.

During the reaction of reverse transcription, mutations are inserted in the sequence of the evolving oligonucleotides which may give rise to more efficient and/or more stable catalytic oligonucleotides.

As can be seen, by utilizing the method of the invention, each catalytically active oligonucleotide remains functional until ligation, splicing, or gap filling occurs, and reverse transcription followed by digestion by RNase H and hence elimination of RNA oligonucleotides takes place only after the catalytic activities commenced. This sequence of events ensures that there is no elimination of functional (i.e. potentially catalytically active) oligonucleotides.

In order to eliminate catalytic oligonucleotides which spontaneously evolve in such a manner so that they contain their own internal promoter and do not require attachment to an external promoter in order to be amplified, it is preferable to construct the substrate oligonucleotide (comprising the promoter) to which the catalytic oligonucleotide is attached, immobilized to a solid support. This can be done, for example, by constructing the oligonucleotide in the substrate oligonucleotide to include biotinylated nucleotides and contacting them with avidin beads. At given intervals during the continuous in vitro evolution method, it is possible to submit the reaction mixture to denaturating conditions, such as an elevated temperatures, in order to separate between oligonucleotide strands bound to each other only by hybridization (and not covalently linked to each other), and then to remove all soluble molecules. This procedure ensures that only catalytic molecules covalently attached (by ligation, gap filling or splicing) to immobilized promoters remain in the reaction mixture while catalytic molecules containing their own internal promoter which did not covalently attach to externally provided promoters but which may be hybridized to the substrate are removed.

In the following, the invention will be described with reference to some non-limiting drawings and examples.

### BRIEF DESCRIPTION OF THE DRAWINGS

FIG. 1 shows a schematic representation of the prior art method for continuous in vitro evolution; and

FIG. 2 shows a schematic representation of the continuous in vitro evolution method of the invention.

### DETAILED DESCRIPTION OF THE INVENTION

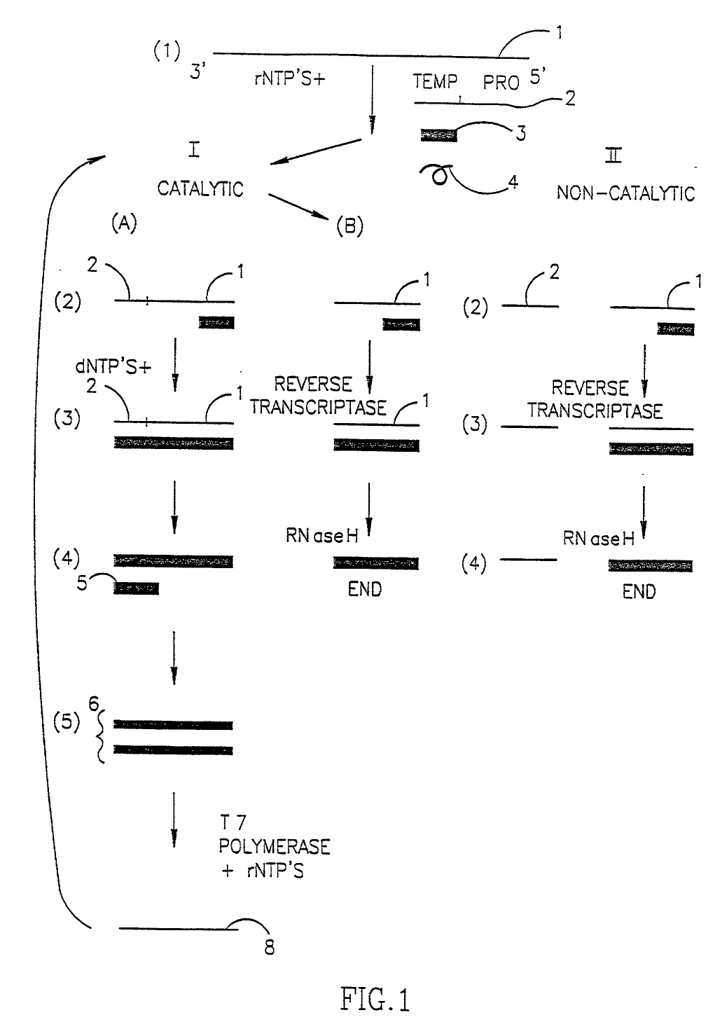

FIG. 1 shows a method for continuous in vitro evolution disclosed in the publication of Wright and Joyce (DNA sequences a thick line; RNA sequences a thin line). A mixture of varying RNA sequences 1 is prepared each sequence being a candidate for being a catalytic oligonucleotide capable of ligation (step 1). The mixture is brought into contact with a reaction mixture comprising RNA promoter sequences capable of being reversed transcribed into a single-stranded template promoter 2 (Temp. Pro); DNA primers for reverse transcription 3; RNase H 4 capable of digesting all RNA strands in RNA/DNA hybrids; and nucleic acid monomers (not shown in the figure). The RNA sequences of 1 fall under one of two classes: catalytic sequences I (left) capable of ligation; or non-catalytic sequences II (right) incapable of ligation.

The non-catalytic sequences II which are not capable of ligating to the RNA promoter 2, hybridize to the DNA primer 3 (step 2). In the presence of reverse transcriptase the primer is elongated to give an RNA/DNA hybrid (step 3), the RNA strand of which is digested by RNase H so that the sequence is eliminated, (step 4) and the reaction ends (END).

The catalytic RNA sequences of class I can proceed via one of two routes: in route (A) (left) ligation precedes reverse transcription and digestion by RNase H (step 2) so that the RNA sequence 1 ligates to the promoter sequence 2 and in the presence of DNA primer 3 and reverse transcriptase is reverse transcripted to DNA (step 3). Then RNase H digests the RNA strand in the RNA/DNA hybrid (step 4). Non-template strands of promoter 5, present in the reaction mixture can then hybridized to the template strand of the promoter to give rise to a ligated construct 6 comprising a functional double-stranded promoter (step 5). In the presence of T₇ RNA polymerase and ribonucleotides, the construct 6 is transcribed to RNA transcript 8 capable of serving again as catalytic molecule I in the method, and so the catalytic RNA sequence is amplified.

However, if the catalytic RNA is reverse transcripted and digested by RNase H prior to ligation (route B-middle), the catalytic RNA which is reversed transcribed (step 3 middle) is digested (step 4 middle) by RNase H before it had a chance to ligate so that it is undesirably eliminated (END). If the balance of the reaction turns to reverse transcription and RNase H digestion (for example since the ligation reaction is slower than the reverse transcription and digestion reactions) all catalytically active RNA sequences will be eliminated and the reaction will come to a dead end (middle route). This is of course undesirable since potentially active sequences are eliminated.

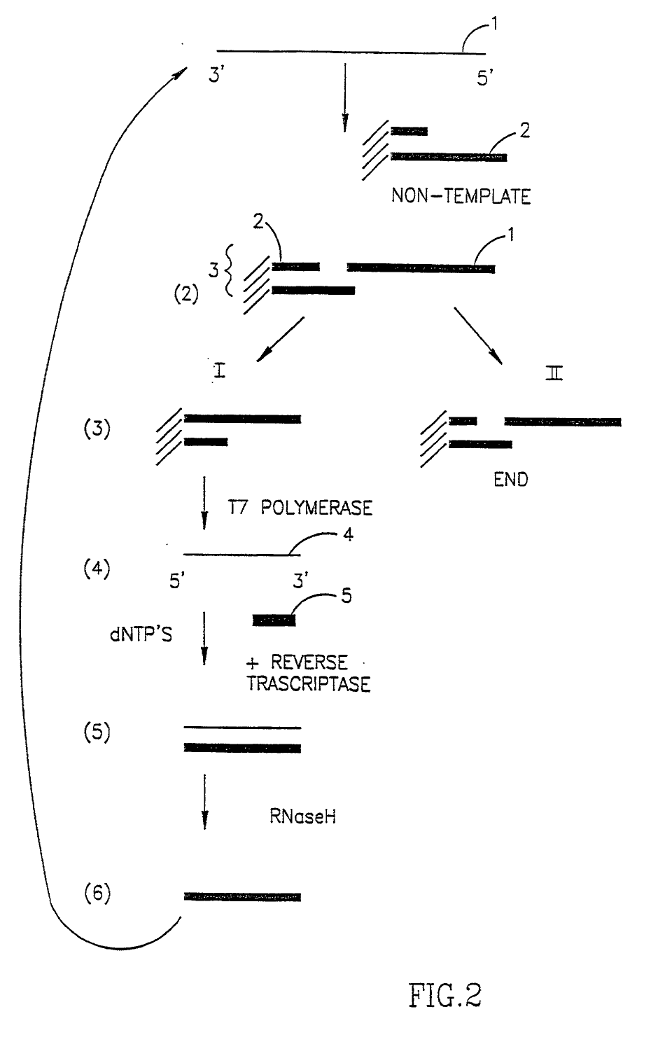

FIG. 2 shows the continuous evolution method of the invention. In step 1, a mixture of DNA oligonucleotides 1 is prepared. Preferably, the 5′ and 3′ terminals of all oligonucleotide species are conserved (indential in all species) while the middle region is random or semi-random. The mixture is brought into contact with a double-stranded DNA promoter 2 which non-template strand comprises a sequence complementary to the (conserved) 3′-end of the oligonucleotide 1.

The constructs 3 produced (step 2) fall under one of two classes: constructs composed of non-catalytic oligonucleotides II (right) in which attachment to promoter 2 (by ligation, splicing or gap filling) did not occur and the reaction ends (END); and constructs containing catalytic oligonucleotide I (left) in which such attachment occurred, resulting in a functional construct (step 3). Functional constructs in the presence of T₇ RNA polymerase produce an RNA transcript 4 (step 4). Said transcript in the presence of a DNA primer 5 (for reverse transcription), reverse transcriptase and deoxy-nucleotides (not shown in the figure) produces an RNA/DNA hybrid 6 (step 5). The RNA strand of hybrid is digested by RNase H (step 6) and the single-stranded DNA thus produced may serve again as the catalytic DNA of 1 and thus may be amplified so that the reaction mixture is gradually enriched with DNA sequences having a desired catalytic activity.

According to the method of the invention reverse transcription and digestion by RNase H cannot commence until attachment of the catalytic oligonucleotide to the promoter by ligation, gap filling or splicing takes place, so that even if these catalytic reactions proceed at a very slow rate, no catalytic oligonucleotide will be eliminated undesirably.

In accordance with the invention, it is preferable that the double-stranded promoter 2 be immobilized, for example, by utilizing biotinated nucleotides which can react with avidin beads (immobilization indicated by diagonal lines). Periodically the reaction mixture is subjected to denaturating conditions (such as elevated temperatures) in order to release those molecules 1 which are merely hybridized to promoter 2 and are not covalently attached to DNA promoter 2 and than all soluble molecules are washed from the reaction mixture. This procedure ensures that only those catalytically active oligonucleotides which were attached to the promoter remain. After such wash the reagent system should be provided again so that the continuous in vitro evolution may proceed. Said immobilization, denaturation and rinsing ensures that catalytic sequences which spontaneously evolve to contain their own internal promoter and thus may be amplified even if not featuring catalytic activities, are discarded.

1. A method for continuous in vitro evolution for obtaining oligonucleotides possessing a catalytic activity of ligation, splicing or gap filling the method comprising:
(a) preparing an oligonucleotide mixture of essentially deoxy-oligo-nucleotides, comprising a plurality of different species of oligonucleotides being candidates for such having said catalytic activity, each species having a different sequence than the other oligonucleotides species;
(b) contacting said mixture with a reagent system comprising:
(b1) oligonucleotides which are substrates for the ligation, splicing, or gap filling catalytic activity, being essentially deoxy oligonucleotides, comprising a functional promoter,
(b2) RNA polymerase,
(b3) ribonucleotide triphosphates of U, A, C and G bases,
(b4) primers for reverse transcription,
(b5) reverse transcriptase,
(b6) deoxy-nucleotide triphosphates of A, T, C and G bases, and
(b7) RNase H, under conditions permitting:
(i) hybridization of complementary oligonucleotides,
(ii) cis ligation, splicing or gap filling, and
(iii) enzymatic activity of RNA polymerase, reverse transcriptase and RNase H;
whereby the reaction mixture becomes enriched with oligonucleotides having said catalytic activity.

2. A method according to claim 1, wherein the oligonucleotides defined in (b1) which are substrates for the ligation, splicing or gap filling activity are immobilized onto a solid support, the method further comprising periodically carrying out the following steps:
(i) subjecting the reaction mixture to conditions enabling denaturation of double-stranded oligonucleotides;
(ii) removing soluble molecules from the reaction mixture; and
(iii) adding the reagent system as defined in claim 1(b).

3. A kit for carrying out a method for continuous in vitro evolution for obtaining oligonucleotides possessing a catalytic activity of ligation, splicing or gap filling the kit comprising:
(a) an oligonucleotide mixture of essentially deoxy-oligonucleotides, comprising a plurality of different species of oligonucleotides being candidates for such having said catalytic activity, each species having a different sequence than the other oligonucleotides species; and
(b) a reagent system comprising:
(b1) oligonucleotides which are substrates for the ligation, splicing, or gap filling catalytic activity, being essentially deoxy oligonucleotides, comprising a functional promoter,
(b2) RNA polymerase,
(b3) ribonucleotide triphosphates of U, A, C and G bases,
(b4) primers for reverse transcription,
(b5) reverse transcriptase,
(b6) deoxy-nucleotide triphosphates of A, T, C and G bases, and
(b7) RNase H.
